(19) United States
(12) Patent Application Publication
(10) Pub. No.: US 2022/0000833 A1
(43) Pub. Date: Jan. 6, 2022

(54) COMPOSITIONS AND METHODS FOR TREATING OBSTRUCTIVE SLEEP APNEA

(71) Applicant: THERAPIX BIOSCIENCES LTD., Givatayim (IL)

(72) Inventors: Adi ZULOFF-SHANI, Ramat HaSharon (IL); Ephraim BRENER, Rishon LeZion (IL); Sscher SHMULEWITZ, Tel Aviv (IL)

(21) Appl. No.: 16/965,548

(22) PCT Filed: Jan. 25, 2019
(86) PCT No.: PCT/IB2019/000073

§ 371 Date: Jul. 28, 2020

Related U.S. Application Data
(60) Provisional application No. 62/623,140, filed on Jan. 29, 2018.

(51) Int. Cl. A61K 31/352; A61K 31/164; A61P 21/00; A61P 11/00

(52) U.S. Cl. CPC A61K 31/352; A61P 11/00; A61P 21/00; A61K 31/164

(57) ABSTRACT

The present disclosure provides pharmaceutical compositions comprising cannabinoids and N-acylethanolamines for treating obstructive sleep apnea (OSA). Also disclosed are methods and kits for treating the disease and minimizing OSA symptoms.

### CROSS REFERENCE TO RELATED APPLICATIONS

This application is based on and claims benefit of priority of U.S. Provisional Patent Application No. 62/623,140, filed on Jan. 29, 2018, which is incorporated herein by reference in its entirety.

### FIELD OF THE INVENTION

The present disclosure relates to certain combinations of cannabinoids and N-acylethanolamines, and to their use in treating diseases, disorders, and conditions of sleep disturbances, such as apnea or their related symptoms. In particular, the present disclosure relates to pharmaceutical compositions and methods for treating obstructive Sleep Apnea.

### BACKGROUND OF THE INVENTION

Deep uninterrupted sleep is a vital factor in human health and takes a third of one's life. Studies indicate that respiratory disturbances affect about 3-7% of men and 2-5% of women with all sleep disorders (Gottlieb, 1999).

Obstructive sleep apnea (OSA) is a common disorder of repetitive pharyngeal collapse during sleep (Malhotra, 2002). Pharyngeal collapse can be complete (causing apnea) or partial (causing hypopnea). Disturbances in gas exchange lead to oxygen desaturation and sleep fragmentation, which contribute to the consequences of OSA—e.g., cardiovascular, metabolic, and neurocognitive effects (Jordan, 2014). Patients with OSA report snoring, witnessed apneas, waking up with a choking sensation, and difficulty initiating or maintaining sleep (Malhotra, 2002). Risk factors include obesity, male sex, age, menopause, fluid retention, family history, and smoking (Jordan, 2014). OSA during sleep may also lead to other morbidities, such as insulin resistance, type II Diabetes Mellitus, stroke, heart disease, neurocognitive disorders, and hypertension (Ip, 2002; Yaggi, 2005; Gottlieb, 2010; Canessa, 2010).

Continuous positive airway pressure (CPAP) device therapy is currently the most common treatment for OSA. Patients wear a face or nasal mask connected to a pump that provides a positive flow of air into the nasal passages to keep the airways open (J. Clin. Sleep Med., 2009). CPAP medical devices have been shown to be effective; however, their use involves an array of side effects. In addition, many OSA sufferers avoid using this machinery because of inconvenience. Thus, improvement of patient adherence to existing treatments and development of new treatments (or combinations of treatments) for OSA are needed.

Earlier reports have suggested the use of either Cannabis or Cannabis-derived chemicals in addressing the symptoms of OSA. One such study demonstrated that treating OSA patients with dronabinol improved or repaired the severity of OSA patient symptoms (Prasad, 2013). However, dronabionol treatment alone may exhibit serious side-effects and may be intolerable at high doses. Therefore, therapeutic regimens that increase the potency, decrease the required dosages, reduce the side effects, and prolong the therapeutic window of cannabinoids, are needed.

Cannabis is a genus of flowering plants from order Rosales, family Cannabaceae, which includes three different species, Cannabis sativa, Cannabis indica, and Cannabis ruderalis, which are indigenous to Central and South Asia (ElSohly, 2007). Cannabis has long been used for hemp fiber, for seed and seed oils, for medicinal purposes, and well as being a recreational drug. Pharmacologically, Cannabis contains 483 known chemical compounds, including at least 85 different cannabinoids (El-Alfy, 2010). Cannabinoids, terpenoids, and other compounds are secreted by glandular trichomes that occur most abundantly on the floral calyxes and bracts of female plants (Mahlberg, 2001).

Cannabinoids are a class of diverse chemical compounds that act on cannabinoid receptors on cells that repress neurotransmitter release in the brain. Cannabinoid receptors are of a class of cell membrane receptors under the G protein-coupled receptor superfamily (Howlett, 2002). As is typical of G protein-coupled receptors, the cannabinoid receptors contain seven transmembrane spanning domains (Sylvaine, 1995). There are two known subtypes of cannabinoid receptors, termed CB1 and CB2, with mounting evidence of more (Matsuda, 1990). The CB1 receptor is expressed mainly in the brain (central nervous system), but also in the lungs, liver, and kidneys. The CB2 receptor is expressed mainly in the immune system and in hematopoietic cells (Pacher, 2011). The protein sequences of CB1 and CB2 receptors are about 44% similar (Latek, 2011).

All classes of cannabinoids derive from cannabigerol-type compounds and differ mainly in the way this precursor is cyclized. The classical cannabinoids are derived from their respective 2-carboxylic acids (2-COOH) by decarboxylation (catalyzed by heat, light, or alkaline conditions). Phytocannabinoids (those derived from the Cannabis plant) include but not limited to: tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV) and cannabigerol monomethyl ether (CBGM). The main way in which the cannabinoids are differentiated is based on their degree of psycho-activity. For example, CBG, CBC, and CBD are not known to be psychologically active agents whereas THC, THCA, CBN, and CBDL along with some other cannabinoids are known to have varying degrees of psycho-activity.

The most notable cannabinoid is the phytocannabinoid Δ9-tetrahydrocannabinol (THC), which is the primary psychoactive component of the cannabis plant. THC has approximately equal affinity for the CB1 and CB2 receptors, and it possesses activities such as an analgesic, psychoactive agent, muscle relaxant, antispasmodic, bronchodilator, neuroprotective, antioxidant, and antipruritic agent.

Dronabinol is the International Nonproprietary Name (INN) for a pure isomer of THC, (−)-Trans-Δ9-tetrahydrocannabinol. Synthesized dronabinol is marketed as Marinol. In the United States, Marinol is a Schedule III drug, available by prescription, considered to be non-narcotic and to have a low risk of physical or mental dependence. Marinol has been approved by the U.S. Food and Drug Administration (FDA) in the treatment of anorexia in AIDS patients, as well as for refractory nausea and vomiting of patients undergoing chemotherapy. An analog of dronabinol, Nabilone, with therapeutic use as an antiemetic and as an adjunct analgesic for neuropathic pain, is available commercially in Canada under the trade name CESAMET. CESAMET has also received FDA approval and began marketing in the U.S. in 2006. Nabilone is a Schedule II drug.

Information about the toxicity of THC is primarily based on results from animal studies. Its toxicity depends on the route of administration and the laboratory animal. The estimated lethal dose of intravenous dronabinol in humans is 30 mg/kg. The adverse effects of THC are mainly psychoactive. It is well established that THC intoxication impairs cognitive functioning on an acute basis, including effecting the ability to plan, organize, solve problems, make decisions, and control impulses. Some studies have suggested that cannabis users have a greater risk of developing psychosis than non-users. In addition, chronic use is associated with elevated levels of apolipoprotein C-III (apoC-III). An increase in apoC-III levels induces the development of hypertriglyceridemia.

Cannabidiol (CBD) is a major phytocannabinoid, accounting for up to 40% of the plant's extract in selected cultivars. CBD is considered to have a wider scope of medical applications than tetrahydrocannabinol (THC). An orally-administered liquid containing CBD has received orphan drug status in the US, for use as a treatment for Dravet syndrome, under the brand name EPIDIOLEX. CBD is able to reduce THC induced cognitive impairment and deficits of visuospatial associative memory. CBD also appears to counteract the sleep-inducing effects of THC. SATIVEX is the first natural cannabis plant derivative to gain full market approval. SATIVEX is a mouth spray for multiple sclerosis (MS) derived neuropathic pain, spasticity, overactive bladder, and other symptoms. Each spray delivers a near 1:1 ratio of CBD to THC, with a fixed dose of 2.7 mg THC and 2.5 mg CBD.

N-acylethanolamines (NAEs) are lipid-derived signaling molecules. They are formed when one of several types of acyl group is linked to the nitrogen atom of ethanolamine (Okamoto, 2004). NAEs are generated by the membrane enzyme NAPE-PLD, and natural bile acids regulate this process (Magotti, 2014).

Anandamide (N-arachidonoylethanolamine, AEA), one of the major components of the endocannabinoid system, is a THC mimetic. Its effects can be either central, in the brain, or peripheral, in other parts of the body, and are mediated primarily by CB1 in the central nervous system, and CB2 in the periphery (Devane, 1992). However, a short half-life due to the action of the enzyme fatty acid amide hydrolase (FAAH) presents a disadvantage for its potential therapeutic use.

Palmitoylethanolamide (PEA, also known as N-(2-hydroxyethyl)hexadecanamide; Hydroxyethylpalmitamide; Palmidrol; N-palmitoylethanolamine; and Palmitylethanolamide) is an endogenous fatty acid amide, belonging to the class of nuclear factor agonists. PEA has been demonstrated to bind to receptors in the cell-nucleus (nuclear receptors) and exert a variety of biological functions related to chronic pain and inflammation. Studies have shown that PEA interacts with distinct non-CB1/CB2 receptors. Studies have also shown that PEA production and inactivation can occur independently of AEA and 2-AG production and inactivation. Much of the biological effects of PEA on cells can be attributed to its affinity to PPAR (O'Sullivan, 2007). PEA also has affinity to cannabinoid-like G-coupled receptors GPR55 and GPR119 as well as the transient receptor potential vanilloid type 1 receptor (TRPV1) (Godlewski, 2009). PEA has anti-inflammatory, anti-nociceptive, neuro-protective, and anti-convulsant properties.

The basic idea of the “entourage effect” is that cannabinoids within the cannabis plant work together, or possess synergy, and affect the body in a mechanism similar to the body's own endocannabinoid system (Ben-Shabata, 1998). This theory serves as the foundation for a relatively controversial idea within pharmacology community, that in certain cases whole plant extractions serve as better therapeutic agents than individual cannabinoid extractions. The “entourage effect” theory has been expanded by Wagner and Ulrich-Merzenich (Wagner, 2009), who define the four basic mechanisms of whole plant extract synergy as follows: (a) ability to affect multiple targets within the body, (b) ability to improve the absorption of active ingredients, (c) ability to overcome bacterial defense mechanisms, and (d) ability to minimize adverse side effects.

There remains a need in the field of OSA management for pharmaceutical combinations of cannabinoids and other agents capable of increasing the potency of the cannabinoids, decreasing the required dosages of the cannabinoids, reducing the side effects of the cannabinoids, and/or prolonging the therapeutic window of cannabinoid action, particularly THC, in humans.

The current discloses a combined therapy of a cannabinoid and an N-acylethanolamines to improve cannabinoid stand-alone induced OSA relief, prolong the therapeutic window of cannabinoids, or reduce the required dose of cannabinoids to achieve desired effects.

### BRIEF DESCRIPTION OF THE DRAWINGS

FIG. 1. Apnea-Hypopnea Index (AHI) for patients 1-4 in Example 1. The AHI is the total number of apneas and hypoapneas that occur divided by the total duration of sleep in hours.

FIG. 2. Average Apnea-Hypopnea Index of patients 1-4 in Example 1.

FIG. 3. Epworth Sleepiness Scale (ESS) for patients 1-4 in Example 1. The ESS is a self-administered questionnaire with eight questions. Respondents are asked to rate, on a 4-point scale (0-3), their usual chances of dozing off or falling asleep while engaged in eight different activities. The ESS score (the sum of the eight question scores, 0-3) can range from 0 to 24. The higher the ESS score, the higher that person's average sleep propensity in daily life.

FIG. 4. Average Epworth Sleepiness Scale of patients 1-4 in Example 1.

FIG. 5. Blood Oxidation Index (ODI) for patients 1-4 in Example 1. The ODI is the number of times per hour of sleep that the blood's oxygen level drop by a certain degree from baseline.

FIG. 6. Average Blood Oxidation Index for patients 1-4 in Example 1.

### SUMMARY OF THE INVENTION

The present disclosure provides methods for treating obstructive sleep apnea (OSA) comprising combinations of cannabinoids and N-acylethanolamines. Also disclosed are pharmaceutical compositions and kits for treating the disease and minimizing OSA symptoms.

The present disclosure is based in part on experimental findings that certain combinations of cannabinoids and N-acylethanolamines enhance the cannabinoid biological activity as a sleep assisting drug and/or reduce its associated side effects.

The present disclosure provides, in one aspect, a method for treating obstructive sleep apnea (OSA) comprising administering to a subject in need thereof a therapeutically-effective amount of at least one cannabinoid or a salt thereof and administering to the subject a therapeutically-effective amount of at least one N-acylethanolamine or a salt thereof, wherein the molar ratio between the cannabinoid and the N-acylethanolamine is between about 1:50 to about 1:500.

In certain embodiments, at least one OSA-related symptom is treated. In certain embodiments, the cannabinoid and the N-acylethanolamine are administered repeatedly until achieving a beneficial change in the condition of the subject according to the Apnea-Hypopnea Index (AHI) index of the subject or Epworth Sleepiness Scale (ESS) questionnaire; compared to the subject's respective score prior to treatment. In certain embodiments, the administration of the cannabinoid and the N-acylethanolamine is repeated until achieving a beneficial change in the condition of the subject according to the positive feedback of the subject compared to his condition prior to treatment.

In certain embodiments, the molar ratio between the cannabinoid and the N-acylethanolamine is between about 1:80 to about 1:320. In certain embodiments, the molar ratio between the cannabinoid and the N-acylethanolamine is between about 1:80 to about 1:160. In certain embodiments, the molar ratio between the cannabinoid and the N-acylethanolamine is between about 1:160 to about 1:320. In certain embodiments, the molar ratio between the cannabinoid and the N-acylethanolamine is 1:80. In certain embodiments, the molar ratio between the cannabinoid and the N-acylethanolamine is 1:160. In certain embodiments, the molar ratio between the cannabinoid and the N-acylethanolamine is 1:320.

In certain embodiments, the therapeutically-effective amount of the cannabinoid or salt thereof is from about 2.5 mg to 10 mg. In certain embodiments, the cannabinoid or salt thereof is administered at about 2.5 mg, 5 mg, or 10 mg. Each possibility represents a separate embodiment of the disclosure. In certain embodiments, the at least one cannabinoid is selected from the group consisting of tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, or any combination thereof. Each possibility represents a separate embodiment of the disclosure. In certain embodiments, the at least one cannabinoid is THC or a salt thereof.

In certain embodiments, the therapeutically-effective amount of the N-acylethanolamine or salt thereof is about 800 mg. In certain embodiments, the at least one N-acylethanolamine or a salt thereof is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, or any combination thereof. Each possibility represents a separate embodiment of the disclosure. In certain embodiments, the N-acylethanolamine is PEA or a salt thereof.

In certain embodiments, the combination comprises THC or a salt thereof and the N-PEA or a salt thereof. In certain embodiments, the cannabinoid and the N-acylethanolamine are administered as a mixture comprising about 0.1-100 mg THC or a salt thereof and about 50-5000 mg PEA or a salt thereof. In certain embodiments, the cannabinoid and the N-acylethanolamine are administered as a mixture comprising about 0.5-50 mg THC or a salt thereof and about 100-2500 mg PEA or a salt thereof. In certain embodiments, the cannabinoid and the N-acylethanolamine are administered as a mixture comprising about 1-25 mg THC or a salt thereof and about 250-2000 mg PEA or a salt thereof. In certain embodiments, the cannabinoid and the N-acylethanolamine are administered as a mixture comprising about 2.5 mg THC or a salt thereof and about 800 mg PEA or a salt thereof. In certain embodiments, the cannabinoid and the N-acylethanolamine are administered as a mixture comprising about 5 mg THC or a salt thereof and about 800 mg PEA or a salt thereof. In certain embodiments, the cannabinoid and the N-acylethanolamine are administered as a mixture comprising about 10 mg THC or a salt thereof and about 800 mg PEA or a salt thereof.

In certain embodiments, the cannabinoid and the N-acylethanolamine described above are formulated for systemic administration. In certain embodiments, the cannabinoid and the N-acylethanolamine are formulated for oral, vaginal, rectal, oral mucosal, sublingual, inhalational, topical, parenteral, intravenous, intramuscular, or subcutaneous administration. In certain embodiments, the cannabinoid and the N-acylethanolamine are formulated for oral, vaginal, or rectal administration. In certain embodiments, the cannabinoid and the N-acylethanolamine are formulated as a solution or as a suppository. Each possibility represents a separate embodiment of the disclosure.

In certain embodiments, the cannabinoid and the N-acylethanolamine are orally administered. In certain embodiments, the cannabinoid and the N-acylethanolamine are daily administered. In certain embodiments, the cannabinoid and the N-acylethanolamine are comprised in the same pharmaceutical composition.

The present disclosure further provides, in another aspect, a method for augmenting the potency of a cannabinoid comprising administering to a subject in need thereof a therapeutically-effective amount of at least one cannabinoid or a salt thereof and administering to the subject a therapeutically-effective amount of at least one N-acylethanolamine or a salt thereof, wherein the molar ratio between the cannabinoid and the N-acylethanolamine is between about 1:50 to about 1:500. In certain embodiments, the method is used to treat obstructive sleep apnea (OSA) or at least one OSA-related symptom.

The present disclosure further provides, in another aspect, a kit for the treatment of obstructive sleep apnea (OSA) comprising a pharmaceutical composition comprising a therapeutically-effective amount of at least one cannabinoid or a salt thereof, a pharmaceutical composition comprising a therapeutically-effective amount of at least one N-acylethanolamine or a salt thereof, and instructions for administering the cannabinoid and N-acylethanolamine, wherein the molar ratio between the cannabinoid and the N-acylethanolamine is between about 1:50 to about 1:500.

In certain embodiments, the methods and kits described above are used to treat a sleep disorder. A sleep disorder, or somnipathy, is a medical disorder of the sleep patterns of a person or animal. Some sleep disorders are serious enough to interfere with normal physical, mental, social and emotional functioning. Polysomnography and actigraphy are tests commonly ordered for some sleep disorders.

In certain embodiments, the sleep disorder is sleep apnea. In certain embodiments, the sleep apnea is obstructive sleep apnea (OSA) or an OSA-related symptom. In certain embodiments, the OSA is caused by complete or partial obstructions of the upper airway. In certain embodiments, it is characterized by repetitive episodes of shallow or paused breathing during sleep, despite the effort to breathe, and is usually associated with a reduction in blood oxygen saturation.

The present disclosure provides, in one aspect, use of a combination therapy for treating obstructive sleep apnea (OSA) in a subject in need thereof, wherein the combination therapy comprises administering to a subject in need thereof a therapeutically-effective amount of at least one cannabinoid or a salt thereof and administering to the subject a therapeutically-effective amount of at least one N-acylethanolamine or a salt thereof, wherein the molar ratio between the cannabinoid and the N-acylethanolamine is between about 1:50 to about 1:500.

In certain embodiments, at least one OSA-related symptom is treated. In certain embodiments, the cannabinoid and the N-acylethanolamine are administered repeatedly until achieving a beneficial change in the condition of the subject according to the Apnea-Hypopnea Index (AHI) index of the subject or Epworth Sleepiness Scale (ESS) questionnaire; compared to the subject's respective score prior to treatment. In certain embodiments, the administration of the cannabinoid and the N-acylethanolamine is repeated until achieving a beneficial change in the condition of the subject according to the positive feedback of the subject compared to his condition prior to treatment.

In certain embodiments, the molar ratio between the cannabinoid and the N-acylethanolamine is between about 1:80 to about 1:320. In certain embodiments, the molar ratio between the cannabinoid and the N-acylethanolamine is between about 1:80 to about 1:160. In certain embodiments, the molar ratio between the cannabinoid and the N-acylethanolamine is between about 1:160 to about 1:320. In certain embodiments, the molar ratio between the cannabinoid and the N-acylethanolamine is 1:80. In certain embodiments, the molar ratio between the cannabinoid and the N-acylethanolamine is 1:160. In certain embodiments, the molar ratio between the cannabinoid and the N-acylethanolamine is 1:320.

In certain embodiments, the therapeutically-effective amount of the cannabinoid or salt thereof is from about 2.5 mg to 10 mg. In certain embodiments, the cannabinoid or salt thereof is administered at about 2.5 mg, 5 mg, or 10 mg. Each possibility represents a separate embodiment of the disclosure. In certain embodiments, the at least one cannabinoid is selected from the group consisting of tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, or any combination thereof. Each possibility represents a separate embodiment of the disclosure. In certain embodiments, the at least one cannabinoid is THC or a salt thereof.

In certain embodiments, the therapeutically-effective amount of the N-acylethanolamine or salt thereof is about 800 mg. In certain embodiments, the at least one N-acylethanolamine or a salt thereof is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, or any combination thereof. Each possibility represents a separate embodiment of the disclosure. In certain embodiments, the N-acylethanolamine is PEA or a salt thereof.

In certain embodiments, the combination comprises THC or a salt thereof and the N-PEA or a salt thereof. In certain embodiments, the cannabinoid and the N-acylethanolamine are administered as a mixture comprising about 0.1-100 mg THC or a salt thereof and about 50-5000 mg PEA or a salt thereof. In certain embodiments, the cannabinoid and the N-acylethanolamine are administered as a mixture comprising about 0.5-50 mg THC or a salt thereof and about 100-2500 mg PEA or a salt thereof. In certain embodiments, the cannabinoid and the N-acylethanolamine are administered as a mixture comprising about 1-25 mg THC or a salt thereof and about 250-2000 mg PEA or a salt thereof. In certain embodiments, the cannabinoid and the N-acylethanolamine are administered as a mixture comprising about 2.5 mg THC or a salt thereof and about 800 mg PEA or a salt thereof. In certain embodiments, the cannabinoid and the N-acylethanolamine are administered as a mixture comprising about 5 mg THC or a salt thereof and about 800 mg PEA or a salt thereof. In certain embodiments, the cannabinoid and the N-acylethanolamine are administered as a mixture comprising about 10 mg THC or a salt thereof and about 800 mg PEA or a salt thereof.

In certain embodiments, the cannabinoid and the N-acylethanolamine described above are formulated for systemic administration. In certain embodiments, the cannabinoid and the N-acylethanolamine are formulated for oral, vaginal, rectal, oral mucosal, sublingual, inhalational, topical, parenteral, intravenous, intramuscular, or subcutaneous administration. In certain embodiments, the cannabinoid and the N-acylethanolamine are formulated for oral, vaginal, or rectal administration. In certain embodiments, the cannabinoid and the N-acylethanolamine are formulated as a solution or as a suppository. Each possibility represents a separate embodiment of the disclosure.

In certain embodiments, the cannabinoid and the N-acylethanolamine are orally administered. In certain embodiments, the cannabinoid and the N-acylethanolamine are daily administered. In certain embodiments, the cannabinoid and the N-acylethanolamine are comprised in the same pharmaceutical composition. The present disclosure further provides, in another aspect, use of a combination therapy for augmenting the potency of a cannabinoid comprising administering to a subject in need thereof a therapeutically-effective amount of at least one cannabinoid or a salt thereof and administering to the subject a therapeutically-effective amount of at least one N-acylethanolamine or a salt thereof, wherein the molar ratio between the cannabinoid and the N-acylethanolamine is between about 1:50 to about 1:500. In certain embodiments, the method is used to treat obstructive sleep apnea (OSA) or at least one OSA-related symptom.

In certain embodiments, at least one OSA-related symptom is treated. In certain embodiments, the cannabinoid and the N-acylethanolamine are administered repeatedly until achieving a beneficial change in the condition of the subject according to the Apnea-Hypopnea Index (AHI) index of the subject or Epworth Sleepiness Scale (ESS) questionnaire; compared to the subject's respective score prior to treatment. In certain embodiments, the administration of the cannabinoid and the N-acylethanolamine is repeated until achieving a beneficial change in the condition of the subject according to the positive feedback of the subject compared to his condition prior to treatment.

In certain embodiments, the molar ratio between the cannabinoid and the N-acylethanolamine is between about 1:80 to about 1:320. In certain embodiments, the molar ratio between the cannabinoid and the N-acylethanolamine is between about 1:80 to about 1:160. In certain embodiments, the molar ratio between the cannabinoid and the N-acylethanolamine is between about 1:160 to about 1:320. In certain embodiments, the molar ratio between the cannabinoid and the N-acylethanolamine is 1:80. In certain embodiments, the molar ratio between the cannabinoid and the N-acylethanolamine is 1:160. In certain embodiments, the molar ratio between the cannabinoid and the N-acylethanolamine is 1:320.

In certain embodiments, the therapeutically-effective amount of the cannabinoid or salt thereof is from about 2.5 mg to 10 mg. In certain embodiments, the cannabinoid or salt thereof is administered at about 2.5 mg, 5 mg, or 10 mg. Each possibility represents a separate embodiment of the disclosure. In certain embodiments, the at least one cannabinoid is selected from the group consisting of tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, or any combination thereof. Each possibility represents a separate embodiment of the disclosure. In certain embodiments, the at least one cannabinoid is THC or a salt thereof.

In certain embodiments, the therapeutically-effective amount of the N-acylethanolamine or salt thereof is about 800 mg. In certain embodiments, the at least one N-acylethanolamine or a salt thereof is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, or any combination thereof. Each possibility represents a separate embodiment of the disclosure. In certain embodiments, the N-acylethanolamine is PEA or a salt thereof.

In certain embodiments, the combination comprises THC or a salt thereof and the N-PEA or a salt thereof. In certain embodiments, the cannabinoid and the N-acylethanolamine are administered as a mixture comprising about 0.1-100 mg THC or a salt thereof and about 50-5000 mg PEA or a salt thereof. In certain embodiments, the cannabinoid and the N-acylethanolamine are administered as a mixture comprising about 0.5-50 mg THC or a salt thereof and about 100-2500 mg PEA or a salt thereof. In certain embodiments, the cannabinoid and the N-acylethanolamine are administered as a mixture comprising about 1-25 mg THC or a salt thereof and about 250-2000 mg PEA or a salt thereof. In certain embodiments, the cannabinoid and the N-acylethanolamine are administered as a mixture comprising about 2.5 mg THC or a salt thereof and about 800 mg PEA or a salt thereof. In certain embodiments, the cannabinoid and the N-acylethanolamine are administered as a mixture comprising about 5 mg THC or a salt thereof and about 800 mg PEA or a salt thereof. In certain embodiments, the cannabinoid and the N-acylethanolamine are administered as a mixture comprising about 10 mg THC or a salt thereof and about 800 mg PEA or a salt thereof.

In certain embodiments, the cannabinoid and the N-acylethanolamine described above are formulated for systemic administration. In certain embodiments, the cannabinoid and the N-acylethanolamine are formulated for oral, vaginal, rectal, oral mucosal, sublingual, inhalational, topical, parenteral, intravenous, intramuscular, or subcutaneous administration. In certain embodiments, the cannabinoid and the N-acylethanolamine are formulated for oral, vaginal, or rectal administration. In certain embodiments, the cannabinoid and the N-acylethanolamine are formulated as a solution or as a suppository. Each possibility represents a separate embodiment of the disclosure.

In certain embodiments, the cannabinoid and the N-acylethanolamine are orally administered. In certain embodiments, the cannabinoid and the N-acylethanolamine are daily administered. In certain embodiments, the cannabinoid and the N-acylethanolamine are comprised in the same pharmaceutical composition

The present disclosure further provides, in another aspect, a method or kit described above for treating pelvic or lower abdominal pain.

Other objects, features and advantages of the present disclosure will become clear from the following description.

### DETAILED DESCRIPTION OF THE INVENTION

The present disclosure provides pharmaceutical compositions and dosage forms, comprising at least one cannabinoid and at least one additional N-acylethanolamine, useful in the treatment of obstructive sleep apnea (OSA). The present disclosure further provides methods for the use of these compositions and dosage forms in treating the diseases or conditions.

OSA is the most common sleep disorder. Cannabinoid-based remedies, such as THC treatment, provide a valuable alternative; however, THC administration may lead to a series of adverse side effects such as impaired cognitive functioning, psychosis, the development of hypertriglyceridemia and an increased risk of myocardial infarction. Thus, there is a great advantage in providing an efficacious cannabinoid-based analgesic therapy with minimal cannabinoid dosages.

The embodiments described herein are based on the discovery that N-acylethanolamine compounds exhibit a cannabinoid-sparing effect. The term “cannabinoid-sparing” as used herein refers to the enablement of the use of low dosages of cannabinoids in instances wherein a mid- or high-dosages of cannabinoids are typically required. The cannabinoid and N-acylethanolamine compounds according to the present disclosure include pharmaceutically acceptable forms thereof, including isomers such as diastereomers and enantiomers, salts, solvates, and polymorphs, as well as racemic mixtures.

According to one aspect, the present disclosure provides a pharmaceutical composition comprising a cannabinoid, N-acylethanolamine, and an acceptable pharmaceutical carrier.

The present disclosure provides, in one aspect, a pharmaceutical composition comprising a therapeutically-effective amount of a mixture of at least one cannabinoid or a salt thereof and at least one N-acylethanolamine or a salt thereof, wherein the molar ratio between the cannabinoid and the N-acylethanolamine is between about 1:50 to about 1:500.

As used herein, a “pharmaceutical composition” refers to a preparation of the active agents described herein with other chemical components such as physiologically suitable carriers and excipients. The purpose of a pharmaceutical composition is to facilitate administration of a compound to an organism. As used herein, the phrase “pharmaceutically acceptable carrier” refers to a carrier, an excipient or a diluent that does not cause significant irritation to an organism and does not abrogate the biological activity and properties of the administered compound. An adjuvant is included under these phrases.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

The term “derivative” as used herein means a compound whose core structure is the same as, or closely resembles that of a reference compound, but which has a chemical or physical modification, such as different or additional side groups.

The term “carrier” as used herein refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The phrase “pharmaceutically acceptable” as used herein refers to molecular entities and compositions that are physiologically tolerable and do not typically produce an allergic or similar toxicity when administered to an individual. Preferably, and particularly where a formulation is used in humans, the term “pharmaceutically acceptable” may mean approved by a regulatory agency (for example, the U.S. Food and Drug Agency) or listed in a generally recognized pharmacopeia for use in animals (e.g., the U.S. Pharmacopeia).

The term “cannabinoid” as used herein generally refers to a class of diverse chemical compounds that act on cannabinoid receptors on cells that repress neurotransmitter release in the brain. Ligands for these receptor proteins include the endocannabinoids (produced naturally in the body by humans and animals), the phytocannabinoids (found in cannabis and some other plants), and synthetic cannabinoids (manufactured artificially). There are at least 85 different cannabinoids isolated from cannabis, exhibiting varied effects (El-Alfy, 2010).

The term “N-acylethanolamine” as used herein generally refers to a type of fatty acid amide, lipid-derived signaling molecules, formed when one of several types of acyl group is linked to the nitrogen atom of ethanolamine. These amides conceptually can be formed from a fatty acid and ethanolamine with the release of a molecule of water, but the known biological synthesis uses a specific phospholipase D to cleave the phospholipid unit from N-acylphosphatidylethanolamines (NAPEs, hormones released by the small intestine into the bloodstream when it processes fat). The suffixes -amine and -amide in these names each refer to the single nitrogen atom of ethanolamine that links the compound together: it is termed “amine” in ethanolamine because it is considered a free terminal nitrogen in that subunit, while it is termed “amide” when it is considered in association with the adjacent carbonyl group of the acyl subunit. Names for these compounds may be encountered with either “amide” or “amine” in the present application. The term “ethanolamine” is used in the generic sense and is meant to include mono-ethanolamine, di-ethanolamine, tri-ethanolamine, and mixtures thereof.

The term “salt” as used herein refers to any form of an active ingredient in which the active ingredient assumes an ionic form and is coupled to a counter ion (a cation or anion) or is in solution. This also includes complexes of the active ingredient with other molecules and ions, in particular, complexes which are formed by ion interaction.

In certain embodiments, the molar ratio between the cannabinoid and the N-acylethanolamine is between about 1:80 to about 1:320. In certain embodiments, the molar ratio between the cannabinoid and the N-acylethanolamine is between about 1:80 to about 1:160. In certain embodiments, the molar ratio between the cannabinoid and the N-acylethanolamine is between about 1:160 to about 1:320. In certain embodiments, the molar ratio between the cannabinoid and the N-acylethanolamine is 1:80. In certain embodiments, the molar ratio between the cannabinoid and the N-acylethanolamine is 1:160. In certain embodiments, the molar ratio between the cannabinoid and the N-acylethanolamine is 1:320.

In certain embodiments, the pharmaceutical composition described above comprises about 2.5-10 mg cannabinoid or a salt thereof. In certain embodiments, the pharmaceutical composition described above comprises about 2.5 mg, about 5 mg, or about 10 mg cannabinoid or a salt thereof. Each possibility represents a separate embodiment of the disclosure. In certain embodiments, the at least one cannabinoid is selected from tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof and any combination thereof. Each possibility represents a separate embodiment of the disclosure. In certain embodiments, the at least one cannabinoid is THC or a salt thereof.

In certain embodiments, the pharmaceutical composition described above comprises about 800 mg N-acylethanolamine or a salt thereof. In certain embodiments, the N-acylethanolamine is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof. Each possibility represents a separate embodiment of the disclosure. In certain embodiments, the N-acylethanolamine is PEA or a salt thereof.

In certain embodiments, the mixture comprises THC or a salt thereof and PEA or a salt thereof. In certain embodiments, the mixture comprises about 0.1-100 mg THC or a salt thereof and about 50-5000 mg PEA or a salt thereof. In certain embodiments, the mixture comprises about 0.5-50 mg THC or a salt thereof and about 100-2500 mg PEA or a salt thereof. In certain embodiments, the mixture comprises about 1-25 mg THC or a salt thereof and about 250-2000 mg PEA or a salt thereof. In certain embodiments, the mixture comprises about 2.5 mg THC or a salt thereof and about 800 mg PEA or a salt thereof. In certain embodiments, the mixture comprises about 5 mg THC or a salt thereof and about 800 mg PEA or a salt thereof. In certain embodiments, the mixture comprises about 10 mg THC or a salt thereof and about 800 mg PEA or a salt thereof.

In certain embodiments, the pharmaceutical composition described above is formulated for systemic administration. In certain embodiments, the pharmaceutical composition described above is formulated for oral, vaginal, rectal, oral mucosal, nasal, sublingual, inhalational, topical, parenteral, intravenous, intramuscular, or subcutaneous administration. In certain embodiments, the pharmaceutical composition described above is formulated for oral, vaginal or rectal administration. In certain embodiments, the pharmaceutical composition described above is formulated as a solution or as a suppository. Each possibility represents a separate embodiment of the disclosure.

The present disclosure further provides, in another aspect, a dosage unit comprising or consisting of any one of the pharmaceutical compositions described above. In some embodiments, the cannanbinoid and the N-acylethanolamine are present in the same pharmaceutical composition. Techniques for formulation and administration of drugs are well known in the art, and may be found, e.g. in “Remington's Pharmaceutical Sciences,” Mack Publishing Co., Easton, Pa.

Pharmaceutical compositions of the present disclosure may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present disclosure may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For topical application, the active ingredients of the pharmaceutical composition may be formulated in cremes, ointments, solutions, patches, sprays, lotions, liniments, varnishes, solid preparations such as silicone sheets, and the like.

The term “topical” as used herein refers to the application of a disclosed composition directly onto at least a portion/region of a subject's skin (human's or non-human's skin) so as to achieve a desired effect, for example, treating dermatological diseases as described herein.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

The term “mucosal administration” relates to delivery of a composition to a mucous membrane, such as the buccal or labial mucosa or the mucosa of the respiratory tract, such as the nasal mucosa.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose (HPMC), and sodium carbomethylcellulose (CMC); and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The term “oral administration” refers to any method of administration in which an active agent can be administered by swallowing, chewing, sucking, or drinking an oral dosage form. Examples of solid dosage forms include conventional tablets, multi-layer tablets, capsules, caplets, etc., which do not substantially release the drug in the mouth or in the oral cavity.

Dragee cores are provided with suitable coatings. For this purpose, concentrated sugar solutions may be used which may optionally contain gum arabic, talc, polyvinyl pyrrolidone, CARBOPOL gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include stiff or soft, sealed capsules made of gelatin and a plasticizer, such as glycerol or sorbitol. The capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal and sublingual administration, the compositions may take the form of tablets or lozenges formulated in conventional manner or in adhesive carriers.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethylcellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The present compositions can also be delivered using an in situ formed depot (ISFD). Examples of in situ formed depots include semi-solid polymers which can be injected as a melt and form a depot upon cooling to body temperature. The requirements for such ISFD include low melting or glass transition temperatures in the range of 25-658° C. and an intrinsic viscosity in the range of 0.05-0.8 dl/g. Below the viscosity threshold of 0.05 dl/g no delayed diffusion could be observed, whereas above 0.8 dl/g the ISFD was no longer injectable using a needle. At temperatures above 378° C. but below 658° C. these polymers behave like viscous fluids which solidify to highly viscous depots. Drugs are incorporated into the molten polymer by mixing without the application of solvents. Thermoplastic pastes (TP) can be used to generate a subcutaneous drug reservoir from which diffusion occurs into the systemic circulation.

In situ cross-linked polymer systems utilize a cross-linked polymer network to control the diffusion of macromolecules over a prolonged period of time. Use of in situ cross-linking implants necessitates protection of the bioactive agents during the cross-linking reaction. This could be achieved by encapsulation into fast degrading gelatin microparticles.

An ISFD can also be based on polymer precipitation. A water-insoluble and biodegradable polymer is dissolved in a biocompatible organic solvent to which a drug is added forming a solution or suspension after mixing. When this formulation is injected into the body the water miscible organic solvent dissipates and water penetrates into the organic phase. This leads to phase separation and precipitation of the polymer forming a depot at the site of injection. One example of such a system is ATRIGELE.

Thermally induced gelling systems can also be used as ISFDs. Numerous polymers show abrupt changes in solubility as a function of environmental temperature. The prototype of a thermosensitive polymer is poly(N-isopropyl acryl amide), poly-NIPAAM, which exhibits a rather sharp lower critical solution temperature.

Thermoplastic pastes such as the new generation of poly(ortho esters) developed by AP Pharma can also be used for depot drug delivery. Such pastes include polymers that are semi-solid at room temperature, hence heating for drug incorporation and injection is no longer necessary. Injection is possible through needles no larger than 22 gauge. The drug can be mixed into the systems in a dry and, therefore, stabilized state. Shrinkage or swelling upon injection is thought to be marginal and, therefore, the initial drug burst is expected to be lower than in the other types of ISFD. An additional advantage is afforded by the self-catalyzed degradation by surface erosion.

The compositions of the present disclosure can also be delivered from medical devices, such as orthopedic implants, contact lenses, micro needle arrays, patches and the like.

Sustained-release (SR), extended-release (ER, XR, or XL), time-release or timed-release, controlled-release (CR), or continuous-release (CR) pills are tablets or capsules formulated to dissolve slowly and release a drug over time. Sustained-release tablets are formulated so that the active ingredient is embedded in a matrix of insoluble substance (e.g. acrylics, polysaccharides, etc.) such that the dissolving drug diffuses out through the holes in the matrix. In some SR formulations the matrix physically swells up to form a gel, so that the drug has first to dissolve in matrix, then exit through the outer surface. The difference between controlled release and sustained release is that controlled release is perfectly zero order release. That is, the drug releases with time irrespective of concentration. On the other hand, sustained release implies slow release of the drug over a time period. It may or may not be controlled release.

Pharmaceutical compositions suitable for use in the context of the present disclosure include compositions wherein the active ingredients are contained in an amount effective to achieve the intended purpose. More specifically, a “therapeutically-effective amount” means an amount of active ingredients effective to prevent, alleviate, or ameliorate symptoms or side effects of a disease or disorder, or prolong the survival of the subject being treated. Determination of a therapeutically effective amount is well within the capability of those skilled in the art, especially in light of the detailed disclosure provided herein.

For any preparation used in the methods of the disclosure, the dosage or the therapeutically effective amount can be estimated initially from in vitro and cell culture assays. For example, a dose can be formulated in animal models to achieve a desired concentration or titer. Such information can be used to more accurately determine useful doses in humans (Reagan-Shaw, 2007).

The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

The dosage of cannabinoid, for example, THC within claimed combination may be ranged from about 0.5 mg to 50 mg THC per subject daily.

The dosage of N-acylethanolamine, for example, PEA within claimed combination may be ranged from about 200 mg to 2100 mg PEA per subject daily.

Continuous daily dosing may not be required; a therapeutic regimen may require cycles, during which time a drug is not administered, or therapy may be provided on an as-needed basis during periods of acute disease worsening.

The present disclosure further provides, in another aspect, a pharmaceutical composition described above, or a dosage unit described above, for use in a method for treating a pelvic or lower abdominal pain.

The term “treating” as used herein, includes, but is not limited to, any one or more of the following: abrogating, ameliorating, inhibiting, attenuating, blocking, suppressing, reducing, delaying, halting, alleviating or preventing one or more symptoms or side effects of the diseases or conditions of the disclosed embodiments.

The present disclosure further provides, in another aspect, a pharmaceutical composition described above, or a dosage unit described above, for use in a method for treating OSA or at least one an OSA-related symptom.

In certain embodiments, the therapeutic potency of the cannabinoid in the pharmaceutical composition is increased compared to the therapeutic potency of the same cannabinoid in a similar pharmaceutical composition without the N-acylethanolamine. In certain embodiments, the required therapeutic dosage of the cannabinoid in the pharmaceutical composition is decreased compared to the required therapeutic dosage of the same cannabinoid in a similar pharmaceutical composition without the N-acylethanolamine. In certain embodiments, at least one side-effect of the cannabinoid in the pharmaceutical composition is reduced compared to the same side-effect of the same cannabinoid in a similar pharmaceutical composition without the N-acylethanolamine. In certain embodiments, the therapeutic window of the cannabinoid in the pharmaceutical composition is expended compared to the therapeutic window of the same cannabinoid in a similar pharmaceutical composition without the N-acylethanolamine.

The present disclosure further provides, in another aspect, a method for treating OSA or at least one OSA-related symptom in a human subject in need thereof, the method comprising the step of administering to the subject a therapeutically-effective amount of a combination of at least one cannabinoid or a salt thereof and at least one N-acylethanolamine or a salt thereof, wherein the molar ratio between the administered cannabinoid and N-acylethanolamine is between about 1:50 to about 1:500, thereby treating the OSA. The cannabinoid and N-acylethanolamine may be comprised in one pharmaceutical composition or in different pharmaceutical compositions.

The present disclosure further provides, in another aspect, a method for augmenting the potency of a cannabinoid, the method comprising the step of administering to the subject a therapeutically-effective amount of a combination of at least one cannabinoid or a salt thereof and at least one N-acylethanolamine or a salt thereof, wherein the molar ratio between the administered cannabinoid and N-acylethanolamine is between about 1:50 to about 1:500, thereby treating the OSA or the at least one OSA-related symptom. The cannabinoid and N-acylethanolamine may be comprised in one pharmaceutical composition or in different pharmaceutical compositions.

Dosage escalation may or may not be required; a therapeutic regimen may require reduction in medication dosage.

Toxicity and therapeutic efficacy of the active ingredients described herein can be determined by standard pharmaceutical procedures in vitro, in cell cultures, or experimental animals The data obtained from these in vitro and cell culture assays and animal studies can be used in formulating a range of dosages for use in human. The dosage may vary depending upon the dosage form employed and the route of administration utilized. The exact formulation, route of administration, and dosage can be chosen by the individual physician in view of the patient's condition (Fingl, 1975).

Depending on the severity and responsiveness of the condition to be treated, dosing can be of a single or a plurality of administrations, with course of treatment lasting from several days to several weeks, or until cure or diminution of the disease state is achieved.

In certain embodiments, the human subject is an obstructive sleep apnea (OSA) patient. In other embodiments, the subject suffers from at least one OSA-related symptom.

Suitable routes of administration may, for example, include oral, rectal, vaginal, topical, nasal, trans-nasal, transmucosal, intestinal, or parenteral delivery, including intramuscular, subcutaneous, and intramedullary injections, as well as intrathecal, direct intraventricular, intravenous, intraperitoneal, intranasal, intraocular injections or by means of inhalation or aspiration (smoking). Alternately, the pharmaceutical composition may be administered locally, rather than in a systemic manner, for example, via injection of the pharmaceutical composition directly into a tissue region of a patient.

In certain embodiments, the cannabinoid and the N-acylethanolamine are orally administered. In certain embodiments, the cannabinoid and the N-acylethanolamine are daily administered. In certain embodiments, the cannabinoid and the N-acylethanolamine are comprised in the same pharmaceutical composition.

In certain embodiments, the administration of the cannabinoid and the N-acylethanolamine is repeated until achieving a beneficial change in the condition of the subject according to (i) Apnea-Hypopnea Index (AHI) index; or (ii) Epworth Sleepiness Scale (ESS) questionnaire; compared to his respective score prior to treatment. In certain embodiments, the administration of the cannabinoid and the N-acylethanolamine is repeated until achieving a beneficial change in the condition of the subject according to the positive feedback of the subject the subject compared to his condition prior to treatment.

Compositions of the present disclosure may, if desired, be presented in a pack or dispenser device, such as an FDA-approved kit, which may contain one or more unit dosage forms containing the active ingredient. The pack may, for example, comprises metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the disclosed embodiments formulated in a pharmaceutically acceptable carrier may also be prepared, placed in an appropriate container, and labeled for treatment of an indicated inflammatory disorder, as further detailed above.

According to an additional aspect, the present disclosure provides a kit comprising:
(i) a pharmaceutical composition comprising a therapeutically-effective amount of at least one cannabinoid or a salt thereof; and
(ii) a pharmaceutical composition comprising a therapeutically-effective amount of at least one N-acylethanolamine or a salt thereof,

wherein the molar ratio between the cannabinoid and the N-acylethanolamine is between about 1:50 to about 1:500.

In certain embodiments, the kit further comprises written instructions for its use in the treatment of obstructive sleep apnea (OSA).

The foregoing description of the specific embodiments will so fully reveal the general nature of the compositions and methods that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

The following examples are presented in order to more fully illustrate some embodiments of the disclosure. They should, in no way be construed, however, as limiting the broad scope of the disclosure. One skilled in the art can readily devise many variations and modifications of the principles disclosed herein without departing from the scope of the disclosure.

### EXAMPLES

### Example 1

### Study to Evaluate Combined Dronabinol and PEA Treatment for OSA

Scientific background: Deep uninterrupted sleep is a vital factor in human health and takes a third of one's life. Based on a previous study, it is speculated that sleep disorders caused by respiratory disturbances constitute around 3-7% in men and 2-5% in women of all sleep disorders.

Obstructive Sleep Apnea (OSA) is characterized by total or partial blockage of the upper respiratory tract for some time intervals during sleep. The clinical reasons for this pathology may result from anatomical or neurological characteristics. Over time, these repetitive blockages may lead to a fall in blood oxygen levels, a noticeable effort to breathe, awakening from sleep, and fatigue. If the OSA does not resolve by itself during the sleep, it usually makes the affected individual wake up. Awaking opens the blockage, then the affected individual returns to the normal breathing. OSA during sleep may also lead to other morbidities, such as insulin resistance, type II Diabetes Mellitus, stroke, ischemic heart disease in men, neurocognitive disorders and hypertension. The main treatment currently in use consists of CPAP device therapy, which provides the patient with air positive pressure to the upper respiratory tract. CPAP medical devices are quite effective, however, their use involves an array of side effects. In addition, many of OSA sufferers avoid using these devices because of inconvenience associated with their use, as well as their side effects. Consequently, the medical needs of these patients are left unmet.

### Palmitoylethanolamide (PEA)

PEA is a lipid organic molecule, which is naturally synthesized by a human and animal body, as well as by some plants. PEA is found (among others) in soy beans, eggs, milk and other food sources. In Europe, PEA is known as a “food for medical purposes”. Following of more than 40 years of clinical research with around 6000 patients and across an array of various clinical indications, PEA was found to be safe, and no side effects has ever been reported. No drug-drug interactions with PEA has been reported so far. The reported dose regimen, when using PEA is ranging between 300 mg and 1200 mg a day for a period of up to two months.

### Dronabinol/Delta-9-Tetrahydrocannabinol (THC)

The active ingredient in Dronabinol is a synthetic THC. THC is known to possess psychoactive abilities, but also for being an analgesic, muscle relaxant, antispasmodic, bronchodilator, neuroprotective, anti-pruritic and antioxidant. THC holds an affinity for both classical cannabinoid receptors (CB1 and CB2). CB1 is widely expressed in various regions of the brain, but has almost no expression in the brain region, which is responsible for breathing. Thus, even when THC is given in overdose, no respiratory depression is observed. The CB2 receptor is expressed mainly on the cells of the immune system.

Dronabinol's safety profile has been extensively described, when a hypothesized lethal dose of Dronabinol in humans is 30 mg/kg (based on rat studies). Serious Dronabinol-associated adverse events (“AEs”) in humans have been reported when the drug has been used at the dose of 0.4 mg/kg. Side effects associated with the use of dronabinol include those involving the cardiovascular system (e.g., increased heart palpitations, dilated blood vessels, facial flushing), digestive system (abdominal pain, nausea, vomiting), but mainly those involving the central system (forgetfulness, anxiety, irritability, ataxia, confusion, loss of personality, dizziness, euphoria, hallucinations, paranoia, sleepiness). Chronic use of Dronabinol in high doses impairs cognitive abilities. In addition, prolonged exposure to high doses of the substance may cause increased levels of Apolipoprotein C-III (apoC-III), which in turn may lead to hypertriglyceridemia.

### Primary Measurements:

Primary safety measurement: Serious Adverse Events (SAEs) report.
Secondary safety measurement: Adverse Events (AEs): anticipated and unanticipated (uAEs).
Primary efficacy measurement: a significant change in AHI index, which assesses the quality of sleep before and after the treatment.
Secondary efficacy measurement: a change in blood oxidation index (ODI) before and after the treatment; improvement in quality assessment index SE; improvement in fatigue and sleepiness index (ESS).

### Study Participants:

30 patients diagnosed with OSA gave their willing consent to participate in the study in accordance with the rules of the Helsinki committee.

### Inclusion and Exclusion Criteria, Termination of Participation in the Study:

### Inclusion Criteria:

| | |
|---|---|
| 1. | OSA diagnosis; OSA severity AHI: 15-30 |
| 2. | Population aged 20-70 |
| 3. | Gender: Males and Females |
| 4. | BMI ≤30 |

### Exclusion Criteria:

| | |
|---|---|
| 1. | Minors |
| 2. | Severity of OSA outside the determined boundaries |
| 3. | Pregnancy |
| 4. | BMI ≥30 |
| 5. | Medical history of narcotic dependency |

### Termination of the Participation in the Experiment:

1. Participant's termination of consent
2. Participant's lack of compliance with the study protocol or participant's failure in taking the drug
3. Participant's unwillingness to continue the treatment due to AEs (mild, moderate or severe, expected and/or unexpected)
4. Investigator's decision to stop administering the study drug due to AEs (mild, moderate or severe, expected and/or unexpected)
5. Participant's lack of compliance during the study or during the follow-up due to various reasons
6. Participant's decision to undergo treatments for sleep disturbances or pain during the study

### Treatment Plan:

PEA was given in tablets orally for the duration of 30 days and was dosed at 800 mg daily (2×400 mg tablets per day) for the whole 30 days of the study.

Dronabinol was given in soft-gel capsules for 30 days in increasing doses of 2.5 mg to 10 mg per day as follows: 2.5 mg per day for the first 3 days, then the dosage was increased to 5 mg per day for the next 3 days, then the dosage was increased to 7.5 mg per day for the next 3 days and finally the dosage was increased to 10 mg per day for remainder of the study.

Each of the subjects was invited to a meeting with a physician in order to undergo clinical evaluation. Every recruited subject underwent a primary sleep examination prior to the beginning of the study and filled in a subjective sleepiness and fatigue questionnaire. Following the basic examination, every recruited patient started to take the study medications in accordance to the study plan detailed above. At the 30 days of treatment, the subjects underwent additional sleep examination/evaluation and filled out the subjective sleepiness and fatigue questionnaire (ESS).

### Clinical Follow-Up Program

During the study, all the subjects in the study were given a telephone and the email address of the study associate for the purpose of reporting the AEs. Once a week, a study associate contacted via phone the subjects and ensured that the subjects were following the treatment plan and that they were not experiencing the AEs. In case and the subject reported the AE to the study associate, the latter reported it to the primary investigator immediately. The primary investigator assessed whether the subject continued taking the study drug and continued his/her participation in the study.

For the duration of the year from the end of the study, all participants received the telephone number and an email address of the investigator as a means of direct contact if needed. After a year since the end of the study, the investigator representative contacted each of the study participants via the phone in order to perform a follow up of the study. In case of reported exceptional AEs, the subject was invited to a physical examination in the study center by the physician.

### Tests During the Study and Follow Up Period:

Sleep assessments at the study center before and after the treatment: the tests were performed by PSG systems (Somnoscreen PSG+, SOMNOmedics GmbH) in compliance with the center protocol for the sleep tests and were performed by a qualified technician. All subjects underwent a pre-treatment and end-of-treatment evaluation to assess the efficacy of the treatment. The scale for determining the improvement was an AHI index, assessing the severity of sleep apnea as measured by sleep testing. In addition, all participants filled out the Epworth Sleepiness Scale (ESS) questionnaire, which measured and quantified the degree of daily fatigue and drowsiness before and after the treatment to evaluate the effect of treatment on the level of subjective fatigue and sleepiness.

### Results:

Four patients completed the study. All completed patients exhibited improvement in examined parameters, including AHI, ESS, and ODI. See Table 1.

TABLE 1

| | First Visit | | | | Last Visit | | | |
|---|---|---|---|---|---|---|---|---|
| Patient | AHI | ESS | ODI | AEs | AHI | ESS | ODI | AEs |
| 1 | 21.6 | 15 | 8.1 | NA* | 13 | 15 | 10.9 | NO** |
| 2 | 23.7 | 0 | 21.9 | NA | 6.7 | 1 | 6.8 | NO |
| 3 | 29.9 | 12 | 19.7 | NA | 3.7 | 7 | 3.7 | NO |
| 4 | 18.6 | 13 | 17.7 | NA | 18.1 | 12 | 18.3 | NO |

NA* and NO** stand for Not Applicable and Not Observed, respectively

### Conclusion:

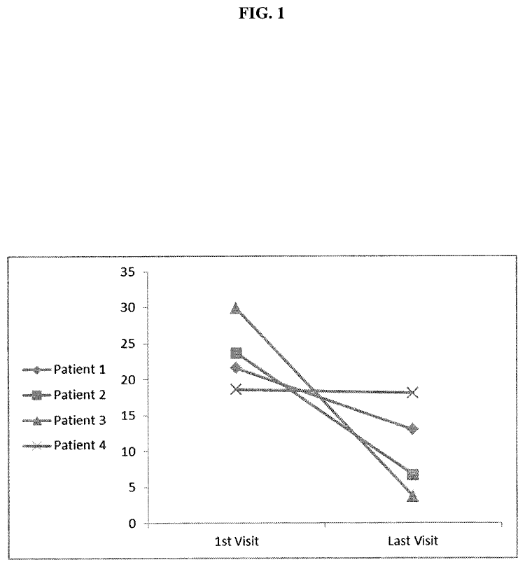

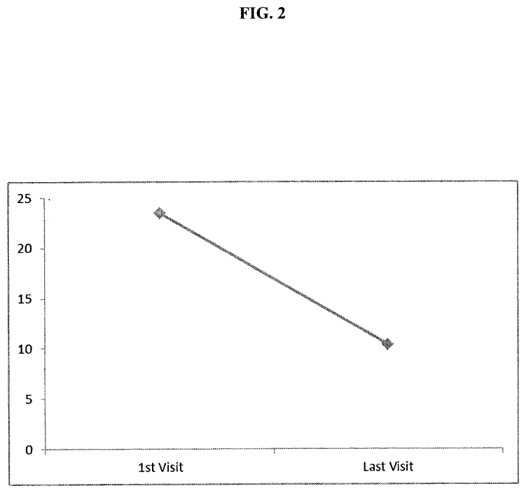

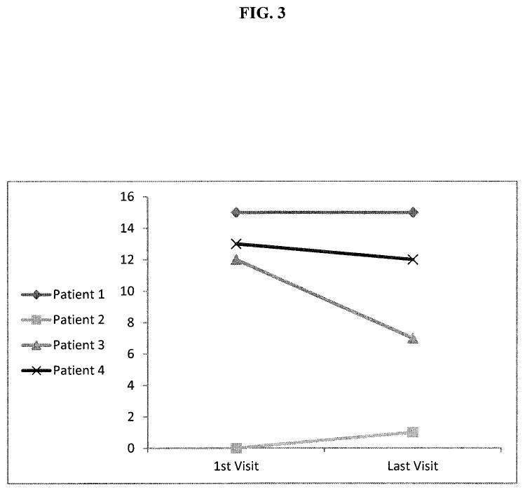

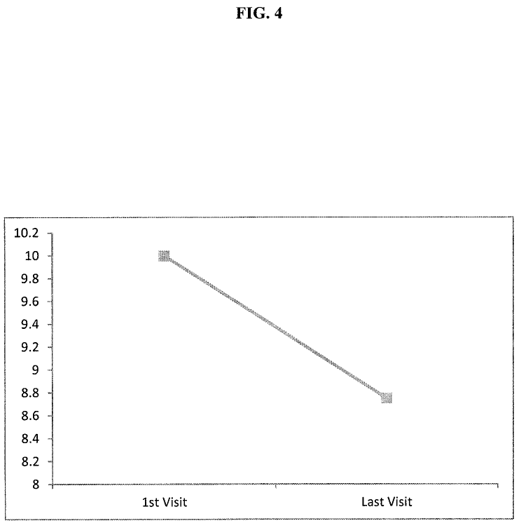

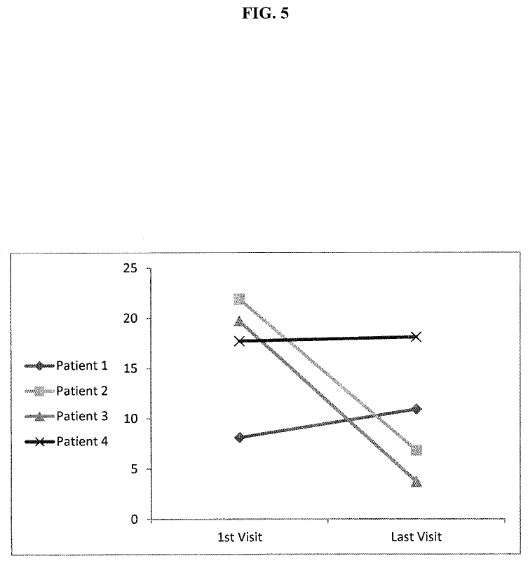

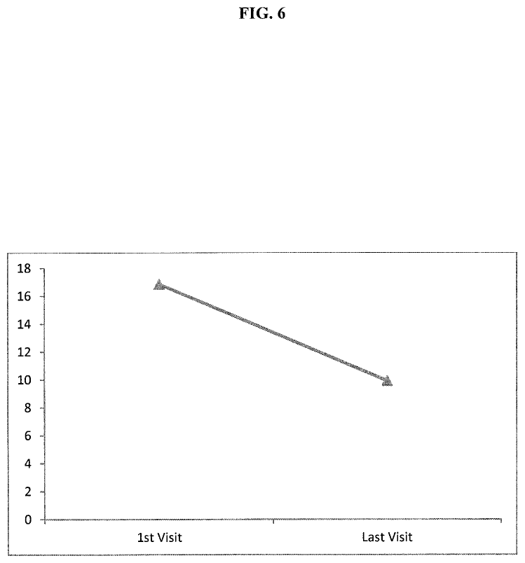

Overall, all measured clinical parameters showed improvement with the combined treatment of Dronabinol 10 mg+PEA 800 mg and Dronabinol 5 mg+PEA 800 mg. The AHI scale, which is the most representative index, when assessing the severity of OSA, demonstrated the most significant progress, in which the initial averaged severity of OSA of 23.45 reduced by almost 56% to 10.375 (See FIGS. 1 and 2). Likewise, the averaged ESS and ODI values were reduced by 12.5% and 41% respectively (See FIGS. 3-6).

1. A method of treating obstructive sleep apnea (OSA) comprising:
administering to a subject in need thereof a therapeutically-effective amount of at least one cannabinoid or a salt thereof; and
administering to the subject a therapeutically-effective amount of at least one N-acylethanolamine or a salt thereof;
wherein the molar ratio between the cannabinoid and the N-acylethanolamine is between about 1:50 to about 1:500.

2. The method of claim 1, wherein at least one OSA-related symptom is treated.

3. The method of claim 1 or 2, wherein the cannabinoid and the N-acylethanolamine are administered repeatedly until achieving a beneficial change in the condition of the subject according to the Apnea-Hypopnea Index (AHI) index of the subject or Epworth Sleepiness Scale (ESS) questionnaire.

4. The method of any of claims 1-3, wherein the molar ratio between the cannabinoid and the N-acylethanolamine is between about 1:80 to about 1:320.

5. The method of any of claims 1-4, wherein the molar ratio between the cannabinoid and the N-acylethanolamine is between about 1:80 to about 1:160.

6. The method of any of claims 1-4, wherein the molar ratio between the cannabinoid and the N-acylethanolamine is between about 1:160 to about 1:320.

7. The method of any of claims 1-4, wherein the molar ratio between the cannabinoid and the N-acylethanolamine is 1:80.

8. The method of any of claims 1-4, wherein the molar ratio between the cannabinoid and the N-acylethanolamine is 1:160.

9. The method of any of claims 1-4, wherein the molar ratio between the cannabinoid and the N-acylethanolamine is 1:320.

10. The method of any of claims 1-9, wherein the therapeutically-effective amount of the cannabinoid or salt thereof is from about 2.5 mg to 10 mg.

11. The method of any of claims 1-10, wherein the cannabinoid or salt thereof is administered at about 2.5 mg, 5 mg, or 10 mg.

12. The method of any of claims 1-11, wherein the at least one cannabinoid is selected from the group consisting of tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, or any combination thereof.

13. The method of any of claims 1-12, wherein the at least one cannabinoid is THC or a salt thereof.

14. The method of any of claims 1-13, wherein the therapeutically-effective amount of the N-acylethanolamine or salt thereof is about 800 mg.

15. The method of any of claims 1-14, wherein the at least one N-acylethanolamine or a salt thereof is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, or any combination thereof.

16. The method of any of claims 1-15, wherein the at least one N-acylethanolamine or a salt thereof is PEA or a salt thereof.

17. The method of any of claims 1-16, wherein the cannabinoid is THC or a salt thereof and the N-acylethanolamine is PEA or a salt thereof.

18. The method of any of claims 1-17, wherein the cannabinoid and the N-acylethanolamine are administered as a mixture comprising about 0.1-100 mg THC or a salt thereof and about 50-5000 mg PEA or a salt thereof.

19. The method of any of claims 1-18, wherein the cannabinoid and the N-acylethanolamine are administered as a mixture comprising about 0.5-50 mg THC or a salt thereof and about 100-2500 mg PEA or a salt thereof.

20. The method of any of claims 1-19, wherein the cannabinoid and the N-acylethanolamine are administered as a mixture comprising about 1-25 mg THC or a salt thereof and about 250-2000 mg PEA or a salt thereof.

21. The method of any of claims 1-20, wherein the cannabinoid and the N-acylethanolamine are administered as a mixture comprising about 2.5 mg THC or a salt thereof and about 800 mg PEA or a salt thereof.

22. The method of any of claims 1-20, wherein the cannabinoid and the N-acylethanolamine are administered as a mixture comprising about 5 mg THC or a salt thereof and about 800 mg PEA or a salt thereof.

23. The method of any of claims 1-20, wherein the cannabinoid and the N-acylethanolamine are administered as a mixture comprising about 10 mg THC or a salt thereof and about 800 mg PEA or a salt thereof.

24. The method of any of claims 1-23, wherein the cannabinoid and the N-acylethanolamine are formulated for systemic administration.

25. The method of any of claims 1-24, wherein the cannabinoid and the N-acylethanolamine are formulated for oral, vaginal, rectal, oral mucosal, sublingual, inhalational, topical, parenteral, intravenous, intramuscular, or subcutaneous administration.

26. The method of any of claims 1-25, wherein the cannabinoid and the N-acylethanolamine are formulated for oral, vaginal, or rectal administration.

27. The method of any of claims 1-26, wherein the cannabinoid and the N-acylethanolamine are formulated as a solution or as a suppository.

28. The method of any of claims 1-27, wherein the cannabinoid and the N-acylethanolamine are formulated as one pharmaceutical composition.

29. A method for augmenting the potency of a cannabinoid comprising:
administering to a subject in need thereof a therapeutically-effective amount of at least one cannabinoid or a salt thereof; and
administering to the subject a therapeutically-effective amount of at least one N-acylethanolamine or a salt thereof;
wherein the molar ratio between the cannabinoid and the N-acylethanolamine is between about 1:50 to about 1:500.

30. The method of claim 29, wherein obstructive sleep apnea (OSA) or at least one OSA-related symptom is treated.

31. The method of claim 29 or 30, wherein the cannabinoid and the N-acylethanolamine are administered repeatedly until achieving a beneficial change in the condition of the subject according to the Apnea-Hypopnea Index (AHI) index of the subject or Epworth Sleepiness Scale (ESS) questionnaire.

32. The method of any of claims 29-31, wherein the molar ratio between the cannabinoid and the N-acylethanolamine is between about 1:80 to about 1:320.

33. The method of any of claims 29-32, wherein the molar ratio between the cannabinoid and the N-acylethanolamine is between about 1:80 to about 1:160.

34. The method of any of claims 29-32, wherein the molar ratio between the cannabinoid and the N-acylethanolamine is between about 1:160 to about 1:320.

35. The method of any of claims 29-32, wherein the molar ratio between the cannabinoid and the N-acylethanolamine is 1:80.

36. The method of any of claims 29-32, wherein the molar ratio between the cannabinoid and the N-acylethanolamine is 1:160.

37. The method of any of claims 29-32, wherein the molar ratio between the cannabinoid and the N-acylethanolamine is 1:320.

38. The method of any of claims 29-37, wherein the therapeutically-effective amount of the cannabinoid or salt thereof is from about 2.5 mg to 10 mg.

39. The method of any of claims 29-38, wherein the cannabinoid or salt thereof is administered at about 2.5 mg, 5 mg, or 10 mg.

40. The method of any of claims 29-38, wherein the at least one cannabinoid is selected from the group consisting of tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, or any combination thereof.

41. The method of any of claims 29-40, wherein the at least one cannabinoid is THC or a salt thereof.

42. The method of any of claims 29-41, wherein the therapeutically-effective amount of the N-acylethanolamine or salt thereof is about 800 mg.

43. The method of any of claims 29-42, wherein the at least one N-acylethanolamine or a salt thereof is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, or any combination thereof.

44. The method of any of claims 29-43, wherein the at least one N-acylethanolamine or a salt thereof is PEA or a salt thereof.

45. The method of any of claims 29-44, wherein the cannabinoid is THC or a salt thereof and the N-acylethanolamine is PEA or a salt thereof.

46. The method of any of claims 29-45, wherein the cannabinoid and the N-acylethanolamine are administered as a mixture comprising about 0.1-100 mg THC or a salt thereof and about 50-5000 mg PEA or a salt thereof.

47. The method of any of claims 29-46, wherein the cannabinoid and the N-acylethanolamine are administered as a mixture comprising about 0.5-50 mg THC or a salt thereof and about 100-2500 mg PEA or a salt thereof.

48. The method of any of claims 29-47, wherein the cannabinoid and the N-acylethanolamine are administered as a mixture comprising about 1-25 mg THC or a salt thereof and about 250-2000 mg PEA or a salt thereof.

49. The method of any of claims 29-48, wherein the cannabinoid and the N-acylethanolamine are administered as a mixture comprising about 2.5 mg THC or a salt thereof and about 800 mg PEA or a salt thereof.

50. The method of any of claims 29-48, wherein the cannabinoid and the N-acylethanolamine are administered as a mixture comprising about 5 mg THC or a salt thereof and about 800 mg PEA or a salt thereof.

51. The method of any of claims 29-48, wherein the cannabinoid and the N-acylethanolamine are administered as a mixture comprising about 10 mg THC or a salt thereof and about 800 mg PEA or a salt thereof.

52. The method of any of claims 29-51, wherein the cannabinoid and the N-acylethanolamine are formulated for systemic administration.

53. The method of any of claims 29-52, wherein the cannabinoid and the N-acylethanolamine are formulated for oral, vaginal, rectal, oral mucosal, sublingual, inhalational, topical, parenteral, intravenous, intramuscular, or subcutaneous administration.

54. The method of any of claims 29-53, wherein the cannabinoid and the N-acylethanolamine are formulated for oral, vaginal, or rectal administration.

55. The method of any of claims 29-54, wherein the cannabinoid and the N-acylethanolamine are formulated as a solution or as a suppository.

56. The method of any of claims 29-55, wherein the cannabinoid and the N-acylethanolamine are formulated as one pharmaceutical composition.

57. A kit for the treatment of obstructive sleep apnea (OSA) comprising:
a pharmaceutical composition comprising a therapeutically-effective amount of at least one cannabinoid or a salt thereof;
a pharmaceutical composition comprising a therapeutically-effective amount of at least one N-acylethanolamine or a salt thereof; and
instructions for administering the cannabinoid and N-acylethanolamine;
wherein the molar ratio between the cannabinoid and the N-acylethanolamine is between about 1:50 to about 1:500.

58. A combination cannabinoid and N-acylethanolamine treatment according to any of claims 1-56 for use in treating a sleep disorder in a patient in need thereof.

59. The method of claim 58, wherein the sleep disorder is obstructive sleep apnea (OSA) or an OSA-related symptom.

60. Use of a combination therapy according to any of claims 1-56 in the manufacture of a medicament for treating a sleep disorder in a subject in need of such treatment.

61. A composition or combination therapy comprising a therapeutically-effective amount of at least one cannabinoid or a salt thereof, and a therapeutically-effective amount of at least one N-acylethanolamine or a salt thereof, for use in a method of treating OSA, wherein the molar ratio between the cannabinoid and the N-acylethanolamine is between about 1:50 to about 1:500.

62. The composition or combination therapy of claim 61, wherein at least one OSA-related symptom is treated.

63. The composition or combination therapy of claim 61 or 62, wherein the cannabinoid and the N-acylethanolamine are administered repeatedly until achieving a beneficial change in the condition of the subject according to the Apnea-Hypopnea Index (AHI) index of the subject or Epworth Sleepiness Scale (ESS) questionnaire.

64. The composition or combination therapy of claims 61-63, wherein the molar ratio between the cannabinoid and the N-acylethanolamine is between about 1:80 to about 1:320.

65. The composition or combination therapy of claims 61-64, wherein the molar ratio between the cannabinoid and the N-acylethanolamine is between about 1:80 to about 1:160.

66. The composition or combination therapy of claims 61-64, wherein the molar ratio between the cannabinoid and the N-acylethanolamine is between about 1:160 to about 1:320.

67. The composition or combination therapy of claims 61-64, wherein the molar ratio between the cannabinoid and the N-acylethanolamine is 1:80.

68. The composition or combination therapy of claims 61-64, wherein the molar ratio between the cannabinoid and the N-acylethanolamine is 1:160.

69. The composition or combination therapy of claims 61-64, wherein the molar ratio between the cannabinoid and the N-acylethanolamine is 1:320.

70. The composition or combination therapy of claims 61-69, wherein the therapeutically-effective amount of the cannabinoid or salt thereof is from about 2.5 mg to 10 mg.

71. The composition or combination therapy of claims 61-70, wherein the cannabinoid or salt thereof is administered at about 2.5 mg, 5 mg, or 10 mg.

72. The composition or combination therapy of claims 61-71, wherein the at least one cannabinoid is selected from the group consisting of tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, or any combination thereof.

73. The composition or combination therapy of claims 61-72, wherein the at least one cannabinoid is THC or a salt thereof.

74. The composition or combination therapy of claims 61-73, wherein the therapeutically-effective amount of the N-acylethanolamine or salt thereof is about 800 mg.

75. The composition or combination therapy of claims 61-74, wherein the at least one N-acylethanolamine or a salt thereof is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, or any combination thereof.

76. The composition or combination therapy of claims 61-75, wherein the at least one N-acylethanolamine or a salt thereof is PEA or a salt thereof.

77. The composition or combination therapy of claims 61-76, wherein the cannabinoid is THC or a salt thereof and the N-acylethanolamine is PEA or a salt thereof.

78. The composition or combination therapy of claims 61-77, wherein the cannabinoid and the N-acylethanolamine are administered as a mixture comprising about 0.1-100 mg THC or a salt thereof and about 50-5000 mg PEA or a salt thereof.

79. The composition or combination therapy of claims 61-78, wherein the cannabinoid and the N-acylethanolamine are administered as a mixture comprising about 0.5-50 mg THC or a salt thereof and about 100-2500 mg PEA or a salt thereof.

80. The composition or combination therapy of claims 61-79, wherein the cannabinoid and the N-acylethanolamine are administered as a mixture comprising about 1-25 mg THC or a salt thereof and about 250-2000 mg PEA or a salt thereof.

81. The composition or combination therapy of claims 61-80, wherein the cannabinoid and the N-acylethanolamine are administered as a mixture comprising about 2.5 mg THC or a salt thereof and about 800 mg PEA or a salt thereof.

82. The composition or combination therapy of claims 61-80, wherein the cannabinoid and the N-acylethanolamine are administered as a mixture comprising about 5 mg THC or a salt thereof and about 800 mg PEA or a salt thereof.

83. The composition or combination therapy of claims 61-80, wherein the cannabinoid and the N-acylethanolamine are administered as a mixture comprising about 10 mg THC or a salt thereof and about 800 mg PEA or a salt thereof.

84. The composition or combination therapy of claims 61-83, wherein the cannabinoid and the N-acylethanolamine are formulated for systemic administration.

85. The composition or combination therapy of claims 61-84, wherein the cannabinoid and the N-acylethanolamine are formulated for oral, vaginal, rectal, oral mucosal, sublingual, inhalational, topical, parenteral, intravenous, intramuscular, or subcutaneous administration.

86. The composition or combination therapy of claims 61-85, wherein the cannabinoid and the N-acylethanolamine are formulated for oral, vaginal, or rectal administration.

87. The composition or combination therapy of claims 61-86, wherein the cannabinoid and the N-acylethanolamine are formulated as a solution or as a suppository.

88. The composition or combination therapy of claims 61-87, wherein the cannabinoid and the N-acylethanolamine are formulated as one pharmaceutical composition.

89. A composition comprising a therapeutically-effective amount of at least one cannabinoid or a salt thereof, and a therapeutically-effective amount of at least one N-acylethanolamine or a salt thereof,
wherein the molar ratio between the cannabinoid and the N-acylethanolamine is between about 1:50 to about 1:500, and wherein the composition augments the potency of the at least one cannabinoid.

90. The composition of claim 89, for use in a method of treating obstructive sleep apnea (OSA) or at least one OSA-related symptom is treated.

91. The composition according to claim 89 or 90, wherein the cannabinoid and the N-acylethanolamine are administered repeatedly until achieving a beneficial change in the condition of the subject according to the Apnea-Hypopnea Index (AHI) index of the subject or Epworth Sleepiness Scale (ESS) questionnaire.

92. The composition according to claim 89-91, wherein the molar ratio between the cannabinoid and the N-acylethanolamine is between about 1:80 to about 1:320.

93. The composition according to claim 89-92, wherein the molar ratio between the cannabinoid and the N-acylethanolamine is between about 1:80 to about 1:160.

94. The composition according to claim 89-92, wherein the molar ratio between the cannabinoid and the N-acylethanolamine is between about 1:160 to about 1:320.

95. The composition according to claim 89-92, wherein the molar ratio between the cannabinoid and the N-acylethanolamine is 1:80.

96. The composition according to claim 89-92, wherein the molar ratio between the cannabinoid and the N-acylethanolamine is 1:160.

97. The composition according to claim 89-92, wherein the molar ratio between the cannabinoid and the N-acylethanolamine is 1:320.

98. The composition according to claim 89-97, wherein the therapeutically-effective amount of the cannabinoid or salt thereof is from about 2.5 mg to 10 mg.

99. The composition according to claim 89-98, wherein the cannabinoid or salt thereof is administered at about 2.5 mg, 5 mg, or 10 mg.

100. The composition according to claim 89-99, wherein the at least one cannabinoid is selected from the group consisting of tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, or any combination thereof.

101. The composition according to claim 89-100, wherein the at least one cannabinoid is THC or a salt thereof.

102. The composition according to claim 89-101, wherein the therapeutically-effective amount of the N-acylethanolamine or salt thereof is about 800 mg.

103. The composition according to claim 89-102, wherein the at least one N-acylethanolamine or a salt thereof is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, or any combination thereof.

104. The composition according to claim 89-103, wherein the at least one N-acylethanolamine or a salt thereof is PEA or a salt thereof.

105. The composition according to claim 89-104, wherein the cannabinoid is THC or a salt thereof and the N-acylethanolamine is PEA or a salt thereof.

106. The composition according to claim 89-105, wherein the cannabinoid and the N-acylethanolamine are administered as a mixture comprising about 0.1-100 mg THC or a salt thereof and about 50-5000 mg PEA or a salt thereof.

107. The composition according to claim 89-106, wherein the cannabinoid and the N-acylethanolamine are administered as a mixture comprising about 0.5-50 mg THC or a salt thereof and about 100-2500 mg PEA or a salt thereof.

108. The composition according to claim 89-107, wherein the cannabinoid and the N-acylethanolamine are administered as a mixture comprising about 1-25 mg THC or a salt thereof and about 250-2000 mg PEA or a salt thereof.

109. The composition according to claim 89-108, wherein the cannabinoid and the N-acylethanolamine are administered as a mixture comprising about 2.5 mg THC or a salt thereof and about 800 mg PEA or a salt thereof.

110. The composition according to claim 89-108, wherein the cannabinoid and the N-acylethanolamine are administered as a mixture comprising about 5 mg THC or a salt thereof and about 800 mg PEA or a salt thereof.

111. The composition according to claim 89-108, wherein the cannabinoid and the N-acylethanolamine are administered as a mixture comprising about 10 mg THC or a salt thereof and about 800 mg PEA or a salt thereof.

112. The composition according to claim 89-111, wherein the cannabinoid and the N-acylethanolamine are formulated for systemic administration.

113. The composition according to claim 89-112, wherein the cannabinoid and the N-acylethanolamine are formulated for oral, vaginal, rectal, oral mucosal, sublingual, inhalational, topical, parenteral, intravenous, intramuscular, or subcutaneous administration.

114. The composition according to claim 89-113, wherein the cannabinoid and the N-acylethanolamine are formulated for oral, vaginal, or rectal administration.

115. The composition according to claim 89-114, wherein the cannabinoid and the N-acylethanolamine are formulated as a solution or as a suppository.

116. The composition according to claim 89-115, wherein the cannabinoid and the N-acylethanolamine are formulated as one pharmaceutical composition.

117. Use of a combination therapy according to any of claims 1-56 in the manufacture of a medicament for treating obstructive sleep apnea (OSA) or an OSA-related symptom in a subject in need of such treatment.
